(19) United States
(12) Patent Application Publication
(10) Pub. No.: US 2009/0306516 A1
(43) Pub. Date: Dec. 10, 2009

(54) ULTRASOUND PROBE AND ULTRASOUND DIAGNOSIS APPARATUS

(75) Inventors: Yutaka OONUKI, Tochigi-ken (JP); Hideki KOSAKU, Tochigi-ken (JP); Yasuhisa MAKITA, Tochigi-ken (JP); Koichi SHIBAMOTO, Tochigi-ken (JP); Minoru AOKI, Tochigi-ken (JP); Tetsuya MOROKAWA, Tochigi-ken (JP)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(73) Assignees: KABUSHIKI KAISHA TOSHIBA, TOKYO (JP); TOSHIBA MEDICAL SYSTEMS CORPORATION, TOCHIGI-KEN (JP)

(21) Appl. No.: 12/480,064

(22) Filed: Jun. 8, 2009

(30) Foreign Application Priority Data
Jun. 9, 2008 (JP) P2008-150568

(51) Int. Cl. A61B 8/14

(52) U.S. Cl. 600/459

(57) ABSTRACT

An ultrasound probe and an ultrasound diagnosis apparatus that can acquire 3-D ultrasound image data of a wide viewing scope including both a diagnosis portion and peripheral portions of the diagnosis portion by swinging a transducer unit in an orthogonal direction to an array of a plurality transducers in the transducer unit without sacrificing the operability of the ultrasound probe.

The ultrasound probe includes a transducer unit having a plurality of transducers, an arm for holding one edge of the transducer unit so as to be extendable in a longitudinal direction, a rail body for holding the other edge of the transducer unit and a driving unit for driving the transducer unit along the rail body. The rail body is comprised of a main rail member having a first curvature in order to form a main track and at least one sub-rail member having a second curvature being larger than the first curvature for forming at least one sub-track.

### CROSS-REFERENCE TO RELATED APPLICATIONS

This application claims priority from, and the benefit of, Japanese Patent Application No. 2008-150568, filed on Jun. 9, 2008, the contents of which are expressly incorporated herein by reference in their entirety.

### BACKGROUND OF THE INVENTION

A. Field of the Invention

The present invention relates to an ultrasound probe and an ultrasound diagnosis apparatus capable of generating three dimensional (3D) ultrasound image data of an object, and more particularly, to an ultrasound probe and an ultrasound diagnosis apparatus capable of acquiring 3-D ultrasound image data of a wide viewing scope including a diagnosis region and periphery regions without sacrificing operability of the ultrasound probe.

B. Background of the Invention

An ultrasound diagnosis apparatus transmits ultrasound pulses from ultrasound transducers (hereinafter “transducers”) installed in a head portion of the ultrasound probe to an object, such as a patient. The transducers receive reflected (echo) ultrasounds that are generated in accordance with differences of acoustic impedances of organs in the object in order to display the organ images on a monitor. Since an ultrasound image diagnosis apparatus can easily obtain and display a two dimensional (2-D) image or a three dimensional (3-D) image of B mode data or color Doppler data in real time by simply touching an ultrasound probe to a patient's body surface, it is widely used as an apparatus for diagnosing the status of a target organ in a patient's body.

Recently, an ultrasound diagnosis apparatus that can generate and display 3-D image data on a monitor by using an ultrasound probe that includes a transducer unit having a plurality of transducers arranged in an array and a mechanical swinging unit for swinging the transducer unit in an orthogonal direction to the array direction of the plurality of transducers has been proposed. (For instance, Japanese Patent Application Publication 2007-6983).

For such a mechanical swinging transducer type ultrasound diagnosis apparatus, it is important to acquire both 3-D ultrasound image data of a diagnostic target portion in an object and also 3-D ultrasound image data of peripheral portions of the diagnostic target portion. Accordingly, it has been required to develop such an ultrasound diagnosis apparatus that can acquire and display 3D ultrasound image data of a wide viewing angle with covering the diagnostic target portion and the periphery portions in a short time.

However, when it is intended to increase a swinging angle of a transducer unit with keeping the same swinging curvature as in the conventional ultrasound probe, it needs to increase a width size of a handling portion of the ultrasound probe in order to cover a wider viewing region. Accordingly, it is inevitable for the ultrasound probe to enlarge the width size of the handling portion of the ultrasound probe in a swing direction. Such an enlargement of the handling portion of the ultrasound probe causes to deteriorate operability of the ultrasound probe. To prevent such a problem for the operability of the ultrasound probe, it needs to reduce a size of a transducer unit provided in the ultrasound probe. To doing so, it is inevitable to reduce a total numbers of the transducers installed in the transducer unit or to reduce a total area of a plurality of transducers in the transducer unit. Such reductions of the total number or the area of the transducers deteriorate the qualities of the acquired and displayed 2-D or 3-D image data due to such the small number or areas of the transducers.

### SUMMARY OF THE INVENTION

To solve the above-mentioned conventional problems and defects, the present invention provides a new ultrasound probe and an ultrasound diagnosis apparatus that can acquire and display 3-D ultrasound image data of a wide viewing region including a diagnostic target portion and the periphery portions in a short time with keeping a good operability of the ultrasound probe.

One aspect of the ultrasound probe consistent with the present invention is an ultrasound probe comprising:

a transducer unit including a plurality of transducers arranged in an array for transmitting and receiving ultrasounds to and from an object; and

a swinging unit configured to swing the transducer unit in an orthogonal direction to the array direction of the plurality of transducers, wherein the swinging unit includes a rail body that is comprised of:

a main rail member having a first curvature for defining a main track for moving the transducer unit; and

at least one sub-rail member having a second curvature that is larger than the first curvature for defining a sub-track for moving the transducer unit, the at least one sub-rail member is connected to the main rail member at an edge connecting portion so as to move the transducer unit along the sub-track that locates an inside of an elongated curvature of the main track extended along a direction to the at least one sub-rail member from the edge connecting portion.

Another aspect of the ultrasound probe consistent with the present invention is an ultrasound probe comprising:

a transducer unit including a plurality of transducers arranged in an array so as to transmit and receive ultrasounds to and from an object; and a swinging unit configured to swing the transducer unit in an orthogonal direction to the array of the plurality of transducers for swinging the transducer unit along a straight main track and at least one curved sub-track member having a prescribed curvature, and connected to an edge of the straight main track.

One aspect of the ultrasound diagnosis system consistent with the present invention is an ultrasound diagnosis apparatus comprising:

a transducer unit configured to transmit and receive ultrasounds to and from an object;

an ultrasound probe having a swinging unit configured to swing the transducer unit along a defined main track having a first curvature and a defined at least one sub-track having a second curvature that is larger than the first curvature, the at least one sub-track is connected to the main track;

an image data generating unit configured to generate 3-D image data in a scope of the main track and the at least one sub-track based on the received ultrasound data through the transducer unit; and

a system control unit configured to control whole operations of each of the units in the ultrasound diagnosis apparatus.

According to the ultrasound probe and the ultrasound diagnosis system consistent with the present invention, it becomes possible to acquire ultrasound image data of a wide viewing angle by swinging the transducer unit provided in the ultrasound probe along the track that is comprised of a center track member having a first curvature and at least one sub-track member having a second curvature that is larger than the first curvature so as to locate the sub-track member inside of a curved extension line of the center track member with preventing a handling size of the ultrasound probe from enlarging. Consequently, the ultrasound diagnosis apparatus consistent with the present invention can improve the examination efficiencies with acquiring a wide angle 3-D ultrasound image data for a diagnosis portion and the periphery without sacrificing the operability of the ultrasound probe.

### BRIEF DESCRIPTION OF THE DRAWINGS

The accompanying drawings, which are incorporated in and constitute part of this specification, illustrate various embodiments and/or features of the present invention, and together with the description, serve to explain embodiments of the present invention. Where possible, the same reference number will be used throughout the drawings to describe the same or like parts. In the drawings:

FIG. 1 is a block diagram illustrating an ultrasound diagnosis apparatus in accordance with a preferred embodiment of the present invention

FIG. 2 illustrates an example structure of the supporting mechanism of the transducer unit in ultrasound the probe shown in FIG. 1.

FIG. 3 illustrates the first embodiment of the swinging structure for swinging the transducer unit in the probe shown in FIG. 2.

FIG. 4 illustrates an example structure of the arm used in the swinging unit shown in FIG. 3.

FIG. 5 illustrates the swinging tracks of the transducer unit shown in FIG. 3.

FIG. 6 is a modified structure of the rail body in the preferred embodiment shown in FIG. 3.

FIG. 7 is a flowchart illustrating acquisitions and displays of 3-D image data by the ultrasound diagnosis apparatus shown in FIG. 1.

FIG. 8 illustrates the swinging angles of the transducer unit shown in FIG. 2 and the directions of the ultrasound transmissions and receptions.

FIG. 9 illustrates the second embodiment of the swinging structure of the transducer unit in the ultrasound probe used for the ultrasound diagnosis apparatus consistent with the present invention.

FIG. 10 illustrates the third embodiment of the swinging structure of the transducer unit in the ultrasound probe used for the ultrasound diagnosis apparatus consistent with the present invention.

### DESCRIPTION OF THE EMBODIMENTS

The accompanying drawings, which are incorporated in and constitute part of this specification, illustrate various embodiments and/or features of the present invention, and together with the description, serve to explain embodiments of the present invention. Where possible, the same reference number will be used throughout the drawings to describe the same or like parts. In the drawings:

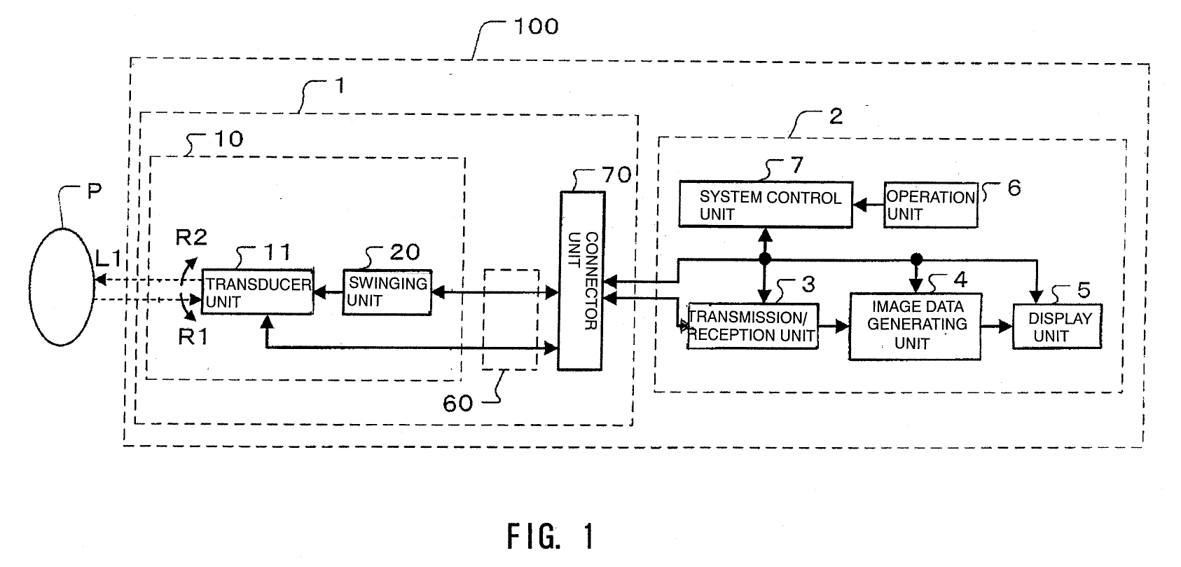

FIG. 1 is a block diagram illustrating a structure of an ultrasound diagnosis apparatus in accordance with a preferred embodiment of the present invention. The ultrasound diagnosis apparatus 100 includes an ultrasound probe 1 for transmitting and receiving ultrasounds to and from an object P and an ultrasound diagnosis apparatus main body 2 for controlling a transducer unit mounted in the ultrasound probe 1.

The ultrasound probe 1 includes a probe unit 10 for transmitting and receiving ultrasounds to and from an object P, a cable unit 60 coupled to the probe unit 10 at one end portion and a connector unit 70 coupled to the other end of the cable unit 60 for carrying the signals of a plurality of channels for the transmission and reception to and from the ultrasound diagnosis apparatus main body 2.

The probe unit 10 includes a prove case comprised of a resin material having a electrical safety and a superior weatherproof and environmental proof characteristics. In the prove unit 10, a transducer unit 11 for transmitting and receiving ultrasounds and a swinging unit 20 for swinging the transducer unit 11 along the curved directions shown by the arrows R1 and R2 are provided. At a tip portion of the probe case, an acoustic widow is provided for transmitting and receiving ultrasounds to and from an object P. The acoustic widow portion (not shown) of the probe case is comprised of a material that has a superior characteristic of ultrasound propagations. An acoustic media AM having a good ultrasound propagation characteristic is filled between the acoustic window of the probe case and the transducer unit 11.

The main body 2 of the ultrasound diagnosis apparatus includes a transmission and reception unit 3 for transmitting ultrasound driving signals to the ultrasound probe 1 and for receiving the reflected ultrasound signals and an image data generating unit 4 for generating three dimensional (3-D) image data based on the two dimensional (2-D) image data generated at a plurality of swinging angles by swinging the transducer unit 11 in the ultrasound probe 1.

The ultrasound diagnosis apparatus main body 2 further includes a display unit 5 for displaying 2-D or 3-D image data generated by the image data generating unit 4, an operation unit 6 for inputting various command signals and a system control unit 7 for totally controlling the swinging unit 20 in the ultrasound probe 1 and the transmission and reception unit 3, the image data generating unit 4 and the display unit 5 in the main body 2.

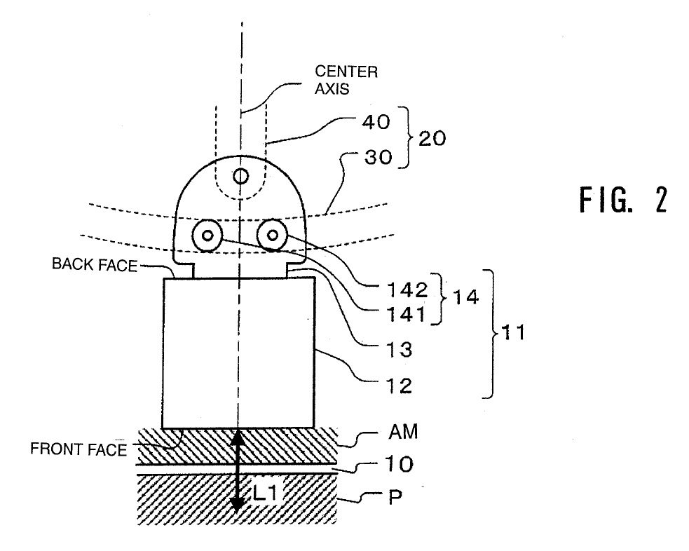

FIG. 2 illustrates a structural relationship of the transducer unit 11 and the swinging unit 20 provided in the ultrasound probe case 19. As illustrated in FIG. 2, the transducer unit 11 includes a transducer body 12, a fixing arm 13 and a roller unit 14 including a first roller 141 and a second roller 142. One end portion of the fixing arm 13 is fixed to a rear surface of the transducer body 12 that is an opposite side of a front surface of the transducer body 12 for transmitting and receiving ultrasounds. The other portion of the fixing arm 13 is rotatably fixed to one portion of the swinging unit 20.

The transducer body 12 includes a plurality (N) of piezoelectric transducers linearly arranged at the front surface portion of the transducer body 12 in order to transmit and receive ultrasounds along a center axis in a direction as shown by an arrow L1. During a transmission time, each of the plurality of piezoelectric transducers converts ultrasound driving signals supplied from the transmission and reception unit 3 in the ultrasound diagnosis main body 2 to transmitting ultrasound pulses. The transmitting ultrasound pulses are transmitted into a body of the object through the acoustic media filled in the probe case 19 and the acoustic window provided at the front surface of the probe case 19. During a reception time, the transducer body 12 converts echo ultrasounds reflected in the object and received through the acoustic window and the acoustic media to ultrasound receiving signals.

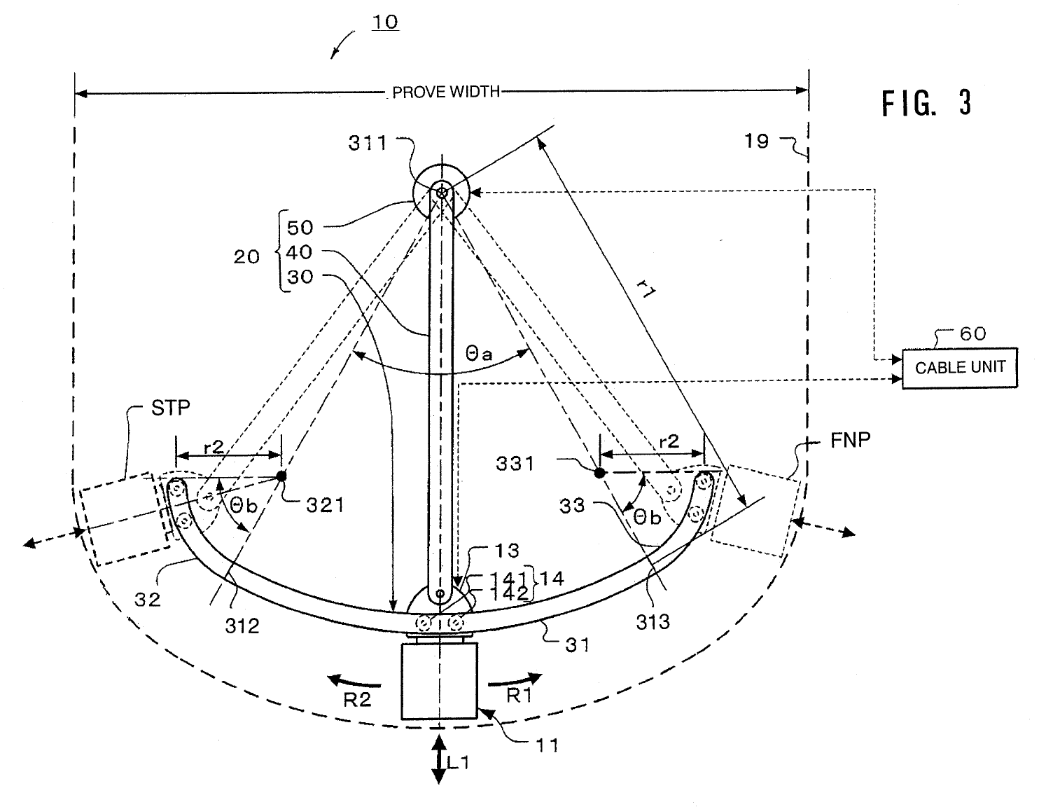

FIG. 3 is the first embodiment structure of the swinging mechanism for the transducer unit 11 shown in FIG. 2. The swinging unit 20 provided in the probe unit 10 includes a rail body 30 for holding the transducer unit 11 so as to be swung, an arm 40 extensible in the L1 direction for swinging the transducer unit 11 along the rail body 30 as the arrow indicated directions R1 and R2 and a driving unit 50 for swinging the arm body 40 in a prescribed angle. As explained later, the arm 40 is extendable in a longitudinal direction. The driving unit 50 is provided at one end portion of the arm 40.

The rail body 30 is comprised of a main rail member 31 and the first and the second sub-rail members 32, 33 that are respectively connected to each edges of the main rail member 31. While the first and the second sub-rail member s 32, 33 are used the embodiment of the present invention, it is also possible to provide at least one sub-rail member for using an ultrasound probe in a particular purpose.

The main rail member 31 is provided at a swinging end portion of the arm 40 so as to form a circular arc configuration based on a radius distance r1 from the swinging center 311 of the arm 40 and a center angle θa of the arm 40. The circular arc shape has a curvature 1/r1 that is represented by an inverse of the radius distance r1. Thus, the main rail member 31 holds and carries the transducer unit 11 so as to swing in a circular arc shape with a curvature 1/r1 around the swinging center 311 of the arm 40.

The first sub-rail member 32 has a circular arc shape that is constructed with a radius of the distance r2 from a hypothetical center 321 that locates on a straight line connecting between one edge part 312 of the main rail member 31 and the swinging center 311 with a center angle θb. The radius distance r2 of the first sub-rail member 32 is smaller than the radius distance r1 of the main rail member 31 (r2<r1). Thus, the first sub-rail member 32 holds and carries the transducer unit 11 so as to swing in a circular arc shape around the hypothetical swinging center 321 with a larger curvature 1/r2 than the curvature 1/r1 of the main rail member 31. In this embodiment, it is supposed that a sweep operation of the transducer unit 11 starts from a swinging start position STP located at an outer edge of the first sub-rail member 32.

Similarly, the second sub-rail member 33 has a circular arc shape that is constructed with a radius of the distance r2 from a hypothetical center 331 that locates on a straight line connecting between other edge part 313 of the main rail member 31 and the swinging center 311 with a center angle θb. The circular arc of the second sub-rail member 33 has a curvature 1/r2 that is represented by an inverse of the radius distance r2. Thus, the second sub-rail member 33 holds and carries the transducer unit 11 so as to swing in a circular arc shape with a curvature 1/r2 around the hypothetical swinging center 331. In this embodiment, it is supposed that a sweep operation of the transducer unit 11 ends at a swinging finish position FNP located at an outer edge of the second sub-rail member 32.

One edge portion of the transducer unit 11 is fixed to, for instance, a couple of railing rollers 141, 142 so as to couple to each of the main rail member 31 and the first and second sub-rail members 32 and 33. By sliding the railing rollers 141 and 142 on each of the rail members, the other edge portion of the transducer unit 11 transmits and receives ultrasounds to and from the outer direction of the rail members, such as shown by an arrow L1.

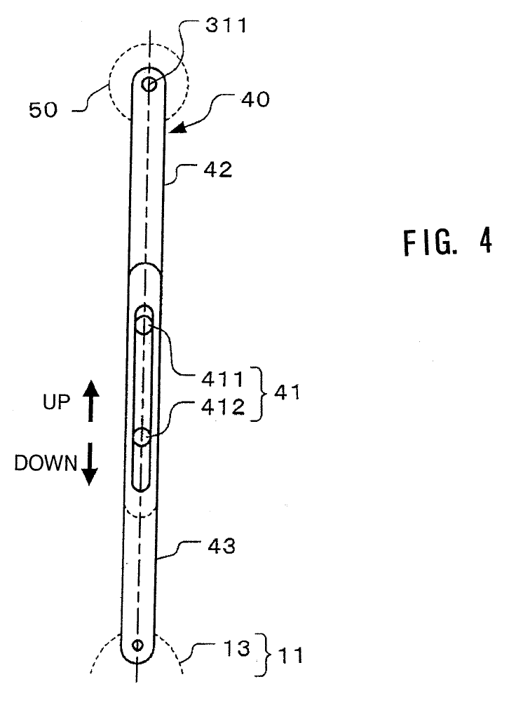

As illustrated in FIG. 4, the arm 40 is comprised of a first arm member 42 and a second arm member 43. One edge portion of the first arm member 42 is fixed to the driving unit as a swinging center 311. The other edge portion of the first arm member 42 holds a couple of arm sliding rollers 411 and 412 that are rotatably provided in a longitudinal direction. One edge portion of the second arm member 43 is coupled to the arm sliding rollers 411 and 412 so as to freely slide along the first arm part 42. The other edge portion of the first arm member 43 holds the fixing arm 13 of the transducer unit 11.

When the transducer unit 11 is placed at a swinging start position STP that locates an outer edge portion of the first sub-rail member 32 as illustrated in FIG. 3, the second arm member 43 slides in the arm center 311 direction so as to contract the arm 40 in the longitudinal direction (an arrow UP direction) through the arm sliding rollers 411 and 412. On the other hand, when the transducers unit 11 passed the sub-rail member 32 and slides along the main rail member 31, the second arm member 43 slides in the fixing arm direction so as to expand the arm 40 in the longitudinal direction (an arrow DOWN direction) through the arm sliding rollers 411 and 412. When transducers unit 11 arrives at the swinging finish position FNP that locates at an edge portion of the second sub-rail member 33 as illustrated in FIG. 3, the transducers unit 11 again slides along the second sub-rail member 33 to the first sub-rail member 32 through the central rail member 31. By repeating these slides between the swinging start position STP and the swinging finish position FNP, the transducers unit 11 swings along the rail 30 with a wide angle.

During when the transducers unit 11 slides on the first and second sub-rail members 32 and 33, the transducer unit 11 moves around each of hypothetical centers 321 or 331 at a curvature 1/r2 with a contacted state of the arm 40 by sliding the second arm 43 to the arm center 311 (or the driving unit 50) direction, respectively. After passing the first sub-rail member 32 or the second sub-rail member 33, when the transducer unit 11 slides along the main rail member 31, the transducer unit 11 moves around the swing center 311 at a curvature 1/r1 with an expanded state of the arm 40 by sliding the second arm 43 to the transducer fixing arm 11 direction (the arrow DOWN direction). Thus, the arm 40 can be contracted or expanded by sliding the second arm member 43 in a longitudinal direction and the transducers unit 11 can repeatedly swing on the different curvature rail members between the swinging start position STP and the swinging finish position FNP.

A rotation axis of the driving unit 50 is located at the swinging center 311 and includes a motor fixed to at one end of the arm 40 and a rotation angle detecting sensor for detecting a rotated angle of the motor, such as a rotary encoder. The swinging angle data detected by the rotation angle detecting sensor is supplied to the system control unit 7 in the ultrasound diagnosis apparatus main body 2. Based on the swinging angle data of the arm 40 supplied from the driving unit 50, the system control unit 7 calculates both a position and a swinging angle of the transducer unit 11 and controls the driving unit 50 based on the calculated position data and the swinging angle data.

Under the controls of the driving unit 50, the transducer unit 11 is accelerated from the swinging start position STP in the R1 direction along the first sub-rail member 32. After moving along the first sub-rail member 32, the transducer unit 11 travels at a constant speed along the main rail member 31. Further, the transducer unit 11 is decelerated along the second edge portion 33 and is stopped at the swinging finish position FNP. Continuously, the transducer unit 11 is decelerated from the swinging finish position FNP in the R2 direction along the second sub-rail member 33 and travels at a constant speed along the main rail member 31. Further, the transducer unit 11 is decelerated along the first sub-rail member 32 and is stopped at the swinging start position STP. By doing so, the transducer unit 11 is swung between the swinging start position STP and the swinging finish position FNP along each of the rail units 31, 32 and 33 in the forth R1 and the back R2 directions.

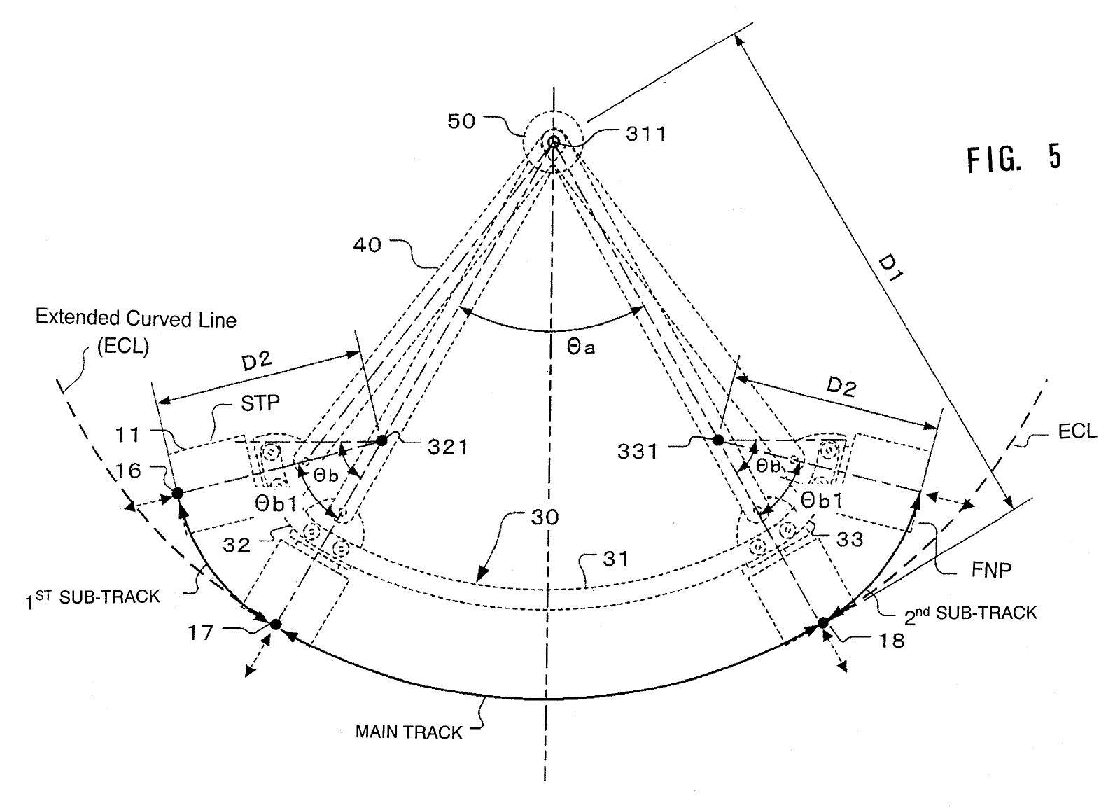

FIG. 5 illustrates a swinging track of the transducer unit 11. During when the transducer unit 11 travels at a constant speed along the main rail member 31, a main track is drawn by the swinging center 311, the center angle θa and a radius D1. The radius D1 is formed by adding a longitudinal length of the transducer body 12 to the distance r1 from the swinging center 311 to the center of the central rail 31. The main track has a first curvature of 1/D1 that is represented by an inverse of the radius D1.

During when the transducers unit 11 swings along the first sub-rail member 32, a first arc-shaped sub-track is determined by a hypothetical swinging center 321, a center angle θb1 that is smaller than the center angle θb and a radius D2 that is decided by adding a distance r2 and a length of the transducers unit body. As illustrated in FIG. 5, the first arc-shaped sub-track locates at an inner-side of a curved extension line (a dotted line) of the center track having the first curvature in a direction to the first sub-track member. Further, the first arc-shaped sub-track has a second curvature (1/D2) that is larger than the first curvature. The sub-track member is connected to the center track member at a connecting edge point 17. Thus, a first tangential line of the main track member having the first curvature at the connecting edge point 17 coincides with a second tangential line of the sub-track member at the same connecting edge point 17.

Similarly, during when the transducer unit 11 swings along the second sub-rail member 33, a second arc-shaped sub-track is generated by determining with a hypothetical swinging center 331, the center angle θb1 and the radius D2. The second arc-shaped sub-track locates at an inner-side of a curved extension line (a dotted line) of the center track in a direction to the second sub-track member, and has a second curvature (1/D2) that is larger than the first curvature. The first tangential line of the main track member having the first curvature at the connecting edge point 18 coincides with the second tangential line of the sub-track member at the connecting edge point 18.

In the embodiments of the present invention, the rail body is constructed by the main rail member and a pair of the sub-rail members that are respectively connected to each outer edge of the main rail member. Of course, it is possible for the rail body to be constructed by connecting at least one sub-rail member to the main rail member.

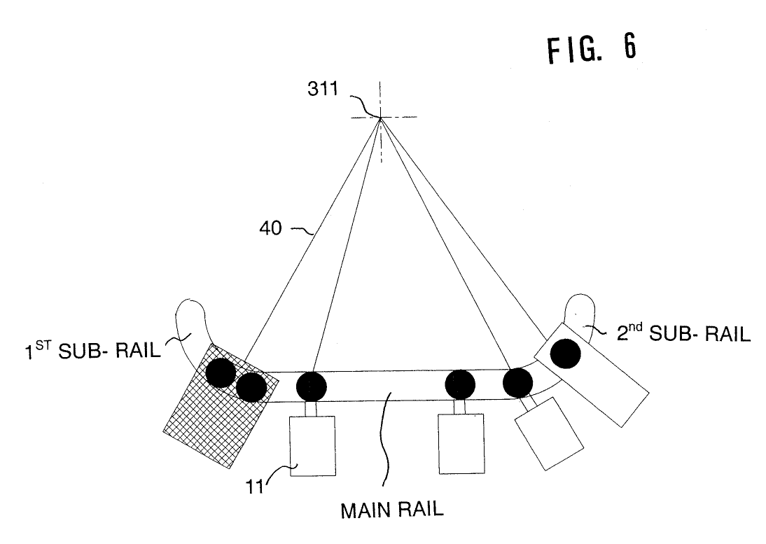

FIG. 6 is a modification of the rail body construction of the first embodiment of the ultrasound probe consistent with the present invention. In this modification, the main rail member 31 shown in FIG. 3 is replaced by a straight rail member 31′ for defining a straight main track for moving the transducer unit, and the first and second sub-rail members 32′ and 33′ are connected to each end of the straight main track member 31′ for constructing curved sub-track members for swinging the transducer unit along a prescribed curvature.

With swinging the transducers unit 11 in the R1 direction or the R2 direction (FIG. 3) along the main track and the first and second sub-tracks, ultrasounds are electronically scanned on the diagnosis portion and the periphery portions by transmitting and receiving ultrasounds in a prescribed time intervals in an orthogonal direction L1 to each of the tracks through the transducers unit 11 under the controls of the transmission/reception unit 3.

As explained the above, the ultrasound probe consistent with the present invention can swing the transducers unit 11 in a wide viewing angle with preventing the width size of the probe unit 10 from enlarging by swinging the transducers unit along the rail body of different curvature tracks.

The system control unit 7 in the ultrasound diagnosis apparatus main body 2 controls the ultrasound probe 1, the transmission/reception unit 3, the image data generating unit 4 and the display unit 5. The driving unit 50 provided in the swinging unit 20 of the ultrasound probe 1 supplies the swinging angle data of the arm 40 to the system control unit 7 through the cable unit 60 and the connector unit 70. The system control unit 7 controls the operation of the driving unit 50 based on the swinging angle data.

The driving unit 50 derives the arm 40 based on the control signals supplied from the system control unit 7 through the connector unit 70 and the carrying cable 60 so as to move the transducers unit 11 to, for instance the swinging start position STP. By driving the arm 40, the transducers unit 11 is swinging between the swinging start position STP and the swinging finish position FNP along the rail 30.

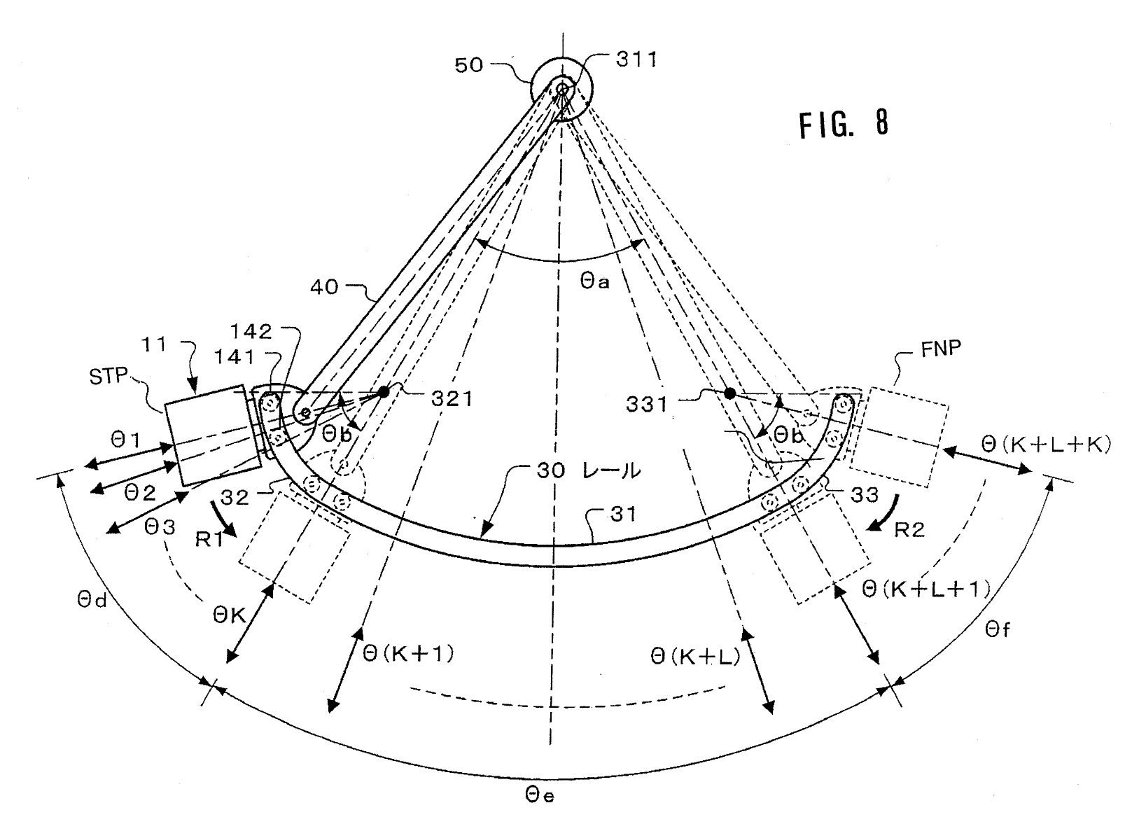

FIG. 8 illustrates the swinging angle of the transducers unit 11 and the directions of the ultrasound transmissions/receptions. The swinging angle of the transducers unit 11 is divided to a main swinging angle θe corresponding to the main track and the first and second edge swinging angles θd and θf respectively corresponding to the first and second sub-tracks. Thus, the first edge swinging angles θd is defined as an angle that the transducer unit 11 slides the first sub-track from the swinging start position STP to a position that a center axis of the arm 40 and a center axis of the transducers unit 11 becomes a straight line. The center swinging angle θe adjoins to the first edge swinging angle θd and is defined so as that the transducer unit 11 swings with keeping the straight line of the center axis of the arm 40 and the center axis of the transducer unit 11. The second edge swinging angle θf adjoins to the center swinging angle θe and is defined as that the transducer unit 11 swings to the swinging finish position FNP with corresponding to the second sub-track.

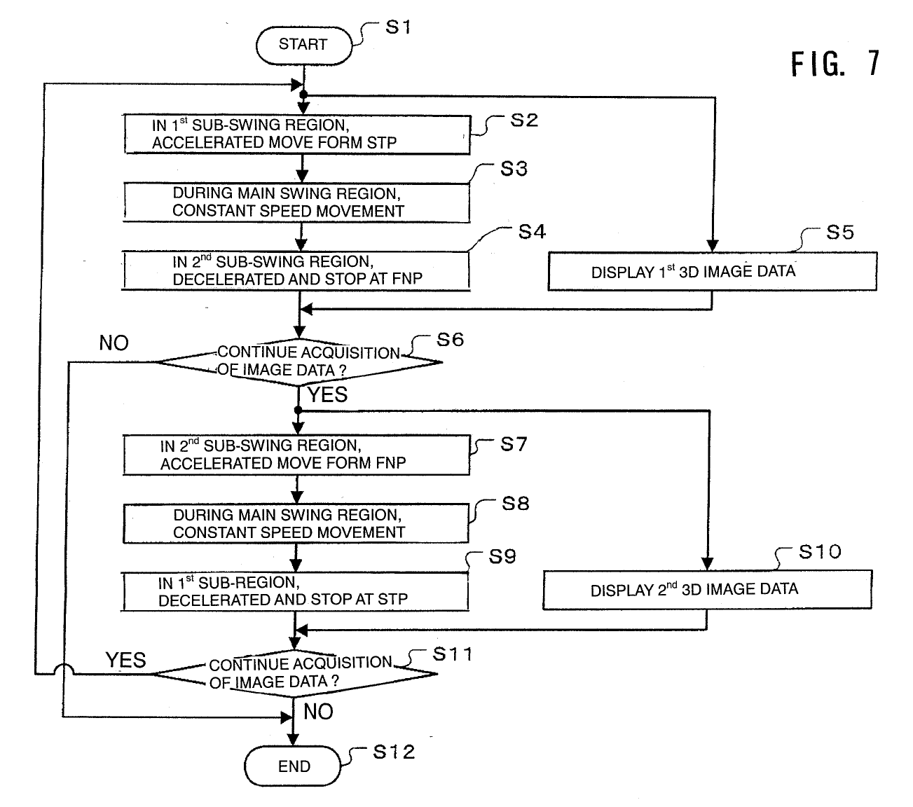

FIG. 7 is a flow chart illustrating the operations of the ultrasound diagnostic apparatus 100. An operator of the ultrasound diagnostic apparatus 100 inputs an object data and sets an image data generating mode, such as a 3-D image (volume) data generating mode through an operation unit 6. After setting such an initial operations, the operator puts a tip portion of the ultrasound probe 1 touching to a body surface of the object P and operates the operation unit 6. By operating the examination start through the operation unit 6, the ultrasound diagnostic apparatus 100 starts an examination (step S1).

When the examination is started, at the first edge swinging scope θd in FIG. 8, the transducer unit 11 starts to swing from the swinging start position STP and moves with accelerating along the first sub-rail member 32 (step S2). Then, the transducer unit 11 moves into the main swinging scope θe, and moves at a constant speed along the main rail member 31 (step S3). During the second edge swinging scope θf, the transducer unit 11 moves along the second sub-rail member 33 with decelerating and stops at the swinging finish position FNP (step S4).

With swinging the transducer unit 11 along each of the rail members, the transmission/reception unit 3 in the ultrasound diagnostic apparatus main body 2 outputs ultrasound driving signals to the transducer unit 11 at a prescribed time intervals based on the control signals from the system control unit 7 through the connector unit 70 and the carrying cable unit 6. Based on the ultrasound driving signals supplied from the transmission/reception unit 3, the transducer unit 11 transmits ultrasounds into the object through an acoustic window of the probe 10 and converts the echo ultrasounds to the receiving signals. The converted ultrasound receiving signals are supplied to the transmission/reception unit 3 through the connector unit 70 and the carrying cable unit 6. The transmission/reception unit 3 processes the ultrasound receiving signals supplied from the transducer unit 11 in order to supply to the image data generating unit 4.

At the swinging start position STP of the first swinging angle, the transducer unit 11 transmits and receives ultrasounds in a depth direction θ1 to the object. Further, the transducer unit 11 scans ultrasounds in the depth direction θ1 and an orthogonal direction to the R1 direction. Based on the receiving signals of the ultrasound scanning in the first depth direction θ1 supplied from the transmission/reception unit 3, the image data generating unit 4 generates 2-D image data. The ultrasound scan data of the depth direction θ1 supplied from the system control unit 7 is stored by affixing to the generated 2-D image data.

Next, when the transducer unit 11 moves to the second swinging angle with accelerating to the R1 direction (FIG. 1) at a prescribed time interval, the ultrasound transmission/reception and scanning are performed in the second depth direction θ2. According to this ultrasound transmission/reception and scanning, the transmission/reception unit 3 supplies the received signals to the image data generating unit 4 in order to generate the second 2-D image data. The generated second 2-D image data is stored with attaching the ultrasound scanning data of the second depth direction θ2.

Since the transducer unit 11 moves with accelerating in the first edge swinging scope θd, each angle intervals between the adjoining depth directions becomes sparsely apart distances at the first swinging angle at the first depth direction θ1 to the k-th angle at the k-th depth direction θk. Corresponded to the ultrasound transmissions/receptions and scans in each of the first depth direction θ1 to the k-th depth direction θk, the K frames data of the first to the k-th 2-D image data generated by the image data generating unit 4 are stored.

The transducer unit 11 moves at a prescribed constant speed in the main swinging scope θe. Accordingly, each of angle intervals between the adjoining depth directions among the (K+1)th depth direction θ (K+1) to the (K+L)th depth direction θ (K+L) becomes an equal distance. This distance is sparsely larger apart than the first edge swinging scope θd. By performing ultrasound transmissions and receptions and also by scanning in each of the (K+1)th depth direction θ(K+1) to the (K+L)th depth direction θ(K+L), the L frames data of the (K+1)th to the (K+L)th 2-D image data generated by the image data generating unit 4 are stored.

Further, in the second edge swinging scope θf, the transducer unit 11 moves with decelerating so as that each angle interval between the adjoining depth directions among the (K+L+1)th depth direction θ(K+L+1) to the (K+L+K)th depth direction θ(K+L+K) becomes gradually closed distances. With corresponding to the ultrasound transmissions/receptions and scans in each of the (K+L+1)th depth direction θ (K+L+1)th to the (K+L+K)th depth direction θ (K+L+K), the K frames data of the first to the k-th 2-D image data generated by the image data generating unit 4 are stored.

Turn to the flowchart in FIG. 6, the image data generating unit 4 generates the first 3-D image data at each of the singing scopes θd, θe and θf from the first 2-D image data to the (K+L+K)th 2-D image data based on each of the scanning data affixed to the generated 2-D image data. The generated 3-D image data is displayed on the display unit 5 (step S5). After the steps S4 and S5, when the examination is continued in order to acquiring another useful 3-D image data (step S6, YES), the operation of the ultrasound diagnostic apparatus 100 successively goes to the steps S7 to S10. When the acquired 3-D image data is sufficient for the examination and an acquisition of 3-D image data does not needed (step S6, NO), the operation goes to step S12 for performing a stop operation of the examination.

By successively performing the ultrasound transmissions/receptions in each of the swinging scopes θd to θe and θf, it can generate and display 3-D image data of a wide viewing scope on the display unit 5. Consequently, it becomes possible to quickly find out a region of interest in the object in a short time.

After the operation step S6, YES, the driving unit 50 swings the transducer unit 11 from the swinging finish position FNP to the swinging start position STP by utilizing the arm 40. Thus, as shown in FIG. 6, during the second edge swinging scope θf, the transducer unit 11 is moved with accelerating (step S7). And in the center swinging scope θe, at a constant speed (step S8). Further, in the first edge swinging scope θd, the transducer unit 11 is moved with decelerating and stops at the swinging start position STP (step S9).

The transmission/reception unit 3 supplies ultrasound driving signals to the transducer unit 11 at a prescribed time intervals. Based on the ultrasound driving signals supplied from the transmission/reception unit 3, the transducers unit 11 transmits ultrasounds into an object P and receives ultrasound echo signals corresponded to the transmitting ultrasounds and supplies the receiving signals to the transmission/reception unit 3. The transmission/reception unit 3 processes the receiving signals and supplies the processed signals to the image data generating unit 4.

As similar to the step S5, the image data generating unit 4 generates the second 3-D image data based on the receiving signals in accordance with the swinging of the transducer unit 11 from the swinging finish position FNP to the swinging start position STP. And the generated second 3-D image data is displayed on the display unit 5 (step S10). After the steps S9 and S10, when the acquired second 3-D image data is not efficient to the examination and another examination is needed (step S11, YES), the operation goes back to the steps S2 and S5. When the acquired second 3-D image data is sufficient for the examination (step S11, NO), the operation goes to the step S12 for stopping the examination.

After the step S6, NO or the step S11, when the operation unit 6 performs an examination ending operation, the system control unit 7 instructs to stop the operations of the ultrasound probe 1, the transmission/reception unit 3, the image data generating unit 4 and the display unit 5. Thus, the ultrasound diagnostic apparatus 100 stops the examination (step S12).

According to this embodiment consistent with the present invention, the transducer unit 11 swings along the rail body 30 that is constructed by the two different curvatures so as to move in a wide angle along the main track and the first and second sub-tracks with preventing the width size of the probe case from enlarging. Further, by performing the ultrasound scans at a plurality of swinging angles in the main track and the first and second sub-tracks, the 3-D image data in a wider viewing scope can be displayed on the display unit 5. Consequently, it becomes possible to increase the efficiencies of the examination by shorting the examination time without sacrificing the operability of the ultrasound probe 1.

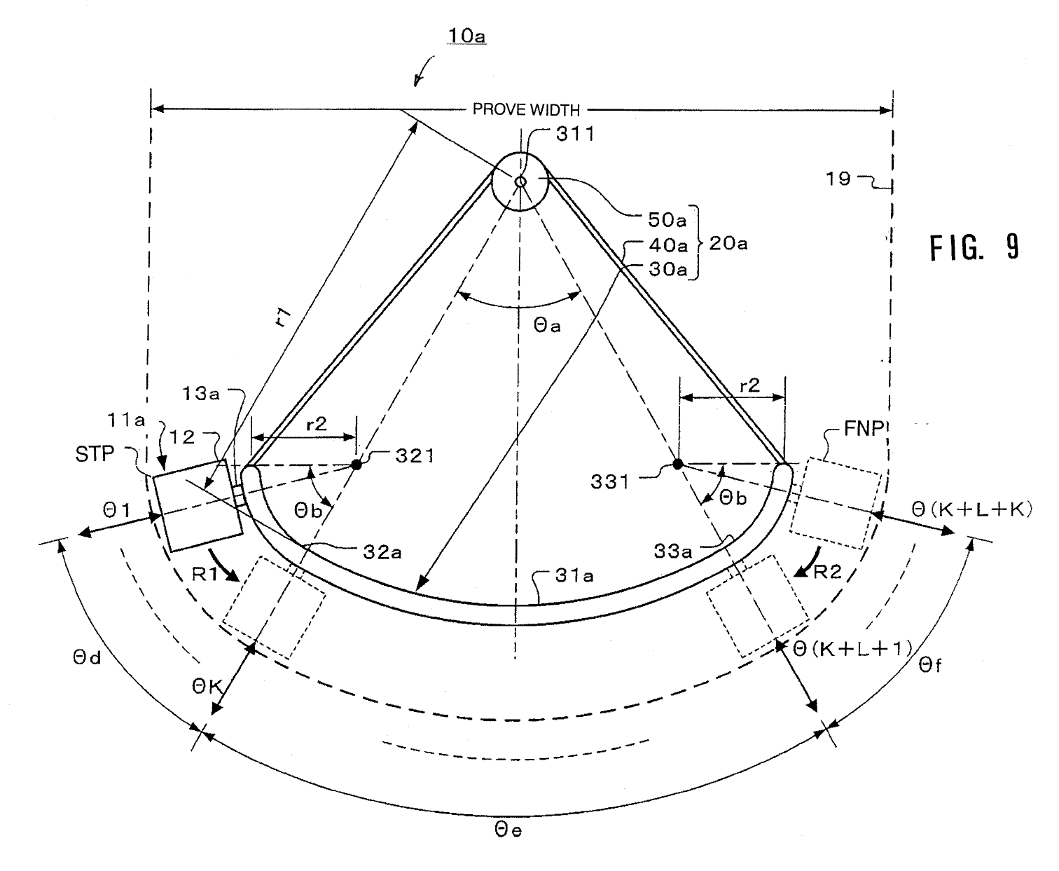

FIG. 9 illustrates the second embodiment of an ultrasound probe 10a for applying to the ultrasound diagnostic apparatus as shown in FIG. 1. The ultrasound probe 10a includes a fixing arm 13a connected to one surface of the transducer main body 12 at one end of the fixing arm 13a and as winging unit 20a holds the other end of the fixing arm 13a. The winging unit 20a includes a curved guide 30a, a belt 40a for holding the transducers unit 11a so as to swing along the curved guide 30a and a driving unit 50a for driving the belt 40a. The curved guide 30a is constructed the same different curvatures as illustrated in FIG. 3.

Thus, the guide body 30a is constructed by a main guide member 31a, a first sub-guide member 32a and a second sub-guide member 33a. The main guide member 31a has the same curvature of the main rail member 31 shown in FIG. 3. And each of the sub-guide members has the same curvature of the first and second sub-rail members 32 and 33 shown in FIG. 3.

The belt 40a surrounds the guide 30a and the driving unit 50a with holding the transducers unit 11a on one portion of the outer surface of the belt.

The main guide 31a can swing the transducer unit 11a around a swinging center at the first circular arc center 311 having the first curvature. The first sub-guide 32a can swing the transducer unit 11a around a swinging center at the second circular arc center 321 having the second curvature. Further, the second sub-guide 33a can swing the transducer unit 11a around a swinging center at the third circular arc center 331 having the second curvature. Accordingly, the transducer unit 11a can be swung along each of the main guide and the first and second sub-guides 32a and 33a and performs ultrasound transmissions/receptions in the orthogonal direction to each of the guides.

The driving unit 50a includes a motor, a pulley fixed to a rotation axis of the motor and a rotation angle detecting sensor for detecting a moving distance of the belt 40a by a rotation of the motor, such as a rotary encoder. The moving distance data of the belt 40a detected by the rotary encoder is supplied to the system control unit 7a in the ultrasound diagnostic apparatus main body 2a.

The system control unit 7a calculates a position and a swinging angle of the transducer unit 11a based on the moved distance data supplied from the driving unit 50a. Based the calculated position data and the swinging angle data, the system control unit 7a controls the driving unit 50a in the ultrasound probe 1a, the transmission/reception unit 3, the image data generating unit 4 and the display unit 5.

As similar to the transducer unit 11 illustrated in FIG. 5, the transducers unit 11a swings along the main track and the first and second tracks with performing the ultrasound transmissions/receptions in an orthogonal direction to each of the tracks. Further, the transducers unit 11a scans ultrasounds in an orthogonal direction to each of the tracks and in an orthogonal direction to each of the R1 or R2 direction.

The driving unit 50a, the transmission/reception unit 3, the image data generating unit 4 and the display unit 5 operate at the similar steps as illustrated in FIG. 6 so as to display the 3-D image data and the second 3-D image data generated by the image data generating unit 4 based on the ultrasound transmissions/receptions through the transducers unit 11a on the display unit 5.

According to the embodiment consistent with the present invention, the guide 30a is constructed by two different curvatures and the transducers unit 11a is swung along the guide 30a. By doing so, the transducers unit 11a can be swung at a wide angle along the plurality of track members with preventing the probe case size from enlarging. Consequently, by scanning ultrasounds at plurality of swinging angles along the plurality of track members, it can generate and display 3-D image data of a wide viewing scope on the display unit 5. Accordingly, it becomes possible to increase the efficiencies of the examination by shorting the examination time without sacrificing the operability of the ultrasound probe 1.

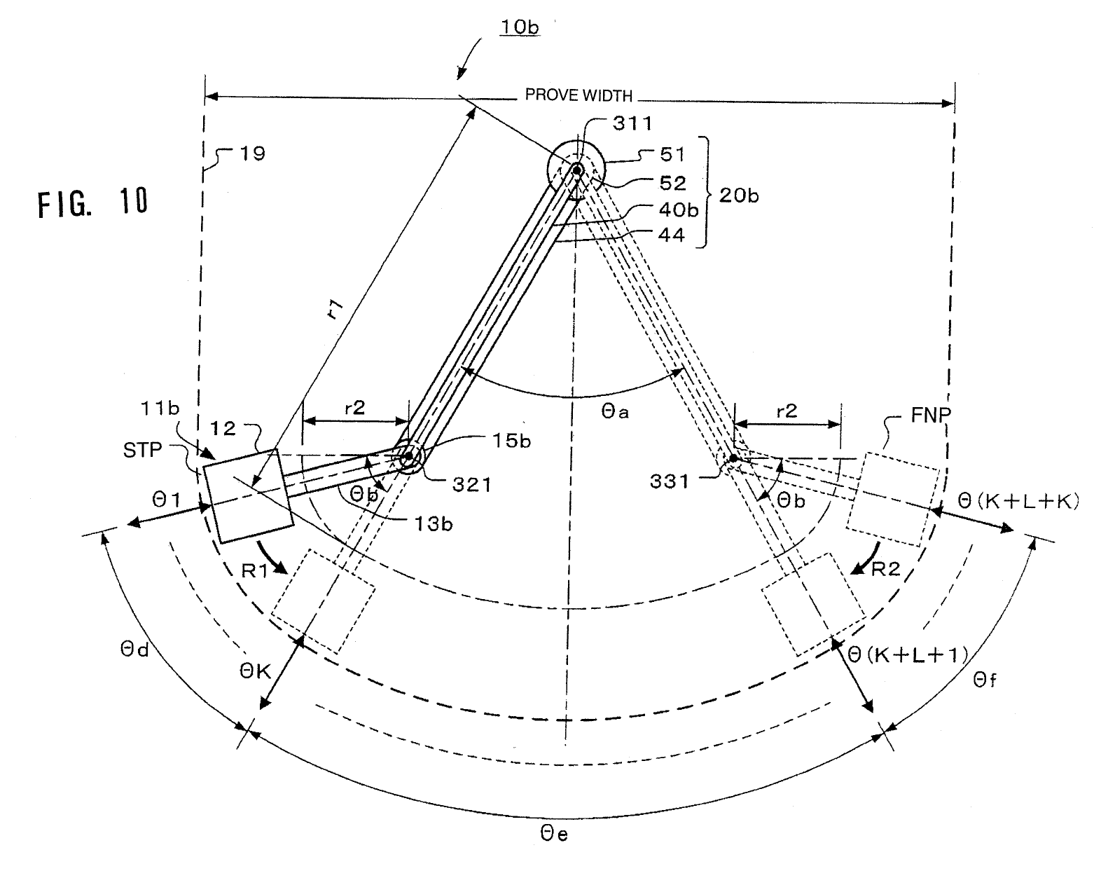

FIG. 10 illustrates the third embodiment of the ultrasound probe 10b for applying to the ultrasound diagnostic apparatus consistent with the present invention. Compared to the embodiment of the diagnostic apparatus 100 illustrated in FIG. 1, the probe unit 10b and the system control unit 7b in the ultrasound diagnostic apparatus main body 2 have different features in the present embodiment of the diagnostic apparatus.

The transducer unit 11b in the probe unit 10b is fixed to one edge portion of an fixing arm at a back surface of the transducer unit body 12. The other edge portion of the fixing arm 13b is coupled to a swinging unit 20b through a pulley 15b at a swinging center of the fixing arm 13b.

The swinging unit 20b includes an arm 40b for swinging a transducer unit 11b within a center swinging scope θe, a first driving unit 51 for swinging the arm 40b, a belt 44 for swinging the transducer unit 11b at a first edge swinging scope θd and a second edge swinging scope θf and a second driving unit 52 for swinging the transducer unit 11b by moving the belt 44 in reciprocating.

One edge portion of the arm 40b in the swinging unit 20b is fixed to the first driving unit 51 at a swinging center 311. The other edge portion of the arm 40b holds the fixing arm 13b for the transducers unit 11b so as to be swung. The belt 44 in the swinging unit 20b is surrounded around a pulley 15b for the transducer unit 11b and the second driving unit 52.

The first driving unit 51 includes a first motor and a first rotation angle detecting sensor. A rotation axis of the first motor is fixed to the one edge portion of the arm 40b. The first rotation angle detecting sensor detects a swinging angle of the arm 40b due to the rotation of the first motor. The swinging angle data of the arm 40b detected by the first rotation angle detecting sensor is supplied to a system control unit 7b in the ultrasound diagnostic apparatus main body 2b.

The second driving unit 52 includes a second motor for rotating a second pulley on which a belt 44 is surrounded and a second rotation angle detecting sensor for detecting a moved distance data of the belt 44 belt 44 due to the rotation of the second motor. The moved distance data of the belt 44 detected by the second rotation angle detecting sensor is supplied to the system control unit 7b in the ultrasound diagnostic apparatus main body 2b.

The system control unit 7b calculates a position and a swinging angle of the transducer unit 11b based on the swinging angle data of the arm 40b and the moved distance data of the belt 44 supplied from the first driving unit 51 and the second driving unit 52. Based on the calculated position data and swinging angle data, the system control unit 7b controls each of the motors in the first and second driving units 51 and 52 provided in the ultrasound probe 1b.

Within the sub-swinging scope θd, the second driving unit 52 drives the belt 44b with fixing the arm 40b at the K-th swinging angle by the first driving unit 5 in order to swing the transducer unit 11b along the main track, as shown in FIG. 5.

Within the main swinging scope θe, the second driving unit 52 holds the belt 44b so as that a center axis of the arm 40b becomes parallel to a center axis of the transducers unit 11b. With keeping this status, the first driving unit 51 swings the arm 40b in order to move the transducer unit 11b along the first sub-track. Further, within the second edge swinging scope θf, the first driving unit 51 keeps the arm 40b at the (K+L+1)th swinging angle. With keeping this status, the second driving unit 52 drives the belt 44b in order to swing the transducers unit 11b along the second sub-track.

With swinging along the main track and the first and second sub-tracks, the transducer unit 11b performs ultrasound transmissions/receptions in an orthogonal direction to each of the tracks. Further, the transducer unit 11b performs ultrasound scans in an orthogonal direction to each of the tracks and also in an orthogonal direction to each of the moving direction R1 or R2.

Under a control of the system control unit 7b, the first and second driving units 51 and 52, the transmission/reception unit 3, the image data generating unit 4 and the display unit 5 are respectively operated as the similar steps shown in FIG. 6. Based on the ultrasound transmissions/receptions through the transducers unit 11b, the image data generating unit 4 generates the first 3-D image data and the second 3-D image data. The generated 3-D image data is displayed on the display unit 5.

According to the present embodiment, in the first sub-swinging scope θd, the transducer unit 11b can be swung so as to draw the first sub-track by fixing the arm 40b at the K-th singing angle. In the main swinging scope θe, the transducer unit 11b can be swung along the main track by keeping the center axis of the transducer unit 11b and the center axis of the arm 40b in parallel. Further, in the second sub-swinging scope θf, the transducer unit 11b can be swung along the second sub-track with keeping the arm 40b at the (K+L+1)th swinging angle. Accordingly, the transducer unit 11a can be swung in a wider angle with preventing the width size of the probe case. Further, by scanning ultrasound at a plurality of swinging angles on each of the main track and the first and second sub-tracks, 3-D image data of a wider viewing scope can be generated and display on the display unit.

As explained the above, the present invention can provide a new ultrasound probe and an ultrasound diagnosis apparatus that can improve the efficiencies of the ultrasound examination by shortening the examination time without scarifying the operability of the ultrasound probe.

Other embodiments consistent with the present invention will be apparent to those skilled in the art from consideration of the specification and practice of the present invention disclosed herein. It is intended that the specification and examples be considered as exemplary only, with the true scope and spirit of the present invention being indicated by the following claims.

What is claimed is:

1. An ultrasound probe comprising:
a transducer unit including a plurality of transducers arranged in an array for transmitting and receiving ultrasounds to and from an object; and
a swinging unit configured to swing the transducer unit in an orthogonal direction to the array direction of the plurality of transducers, wherein the swinging unit includes a rail body that is comprised of:
a main rail member having a first curvature for defining a main track for moving the transducer unit; and
at least one sub-rail member having a second curvature that is larger than the first curvature for defining a sub-track for moving the transducer unit, the at least one sub-rail member is connected to the main rail member at an edge connecting portion so as to move the transducer unit along the sub-track that locates an inside of an elongated curvature of the main track extended along a direction to the at least one sub-rail member from the edge connecting portion.

2. The ultrasound probe according to claim 1, wherein a first tangential line of the main track having the first curvature at the edge connecting portion coincide with a second tangential line of the sub-track at the edge connecting portion.

3. The ultrasound probe according to claim 1, wherein
the swinging unit includes the rail body comprised of the main rail member for moving the transducer unit along the main track having the first curvature and the at least one sub-rail member for moving the transducer unit along the sub-track having the second curvature that is larger than the first curvature;
an extendable arm for swinging the transducer unit in an longitudinal direction to each of the main track and the at least one sub-track; and
a driving unit for driving the arm;
whereby the transducer unit performs ultrasound transmissions/receptions in an orthogonal direction along each of the main track and the at least one sub-track.

4. The ultrasound probe according to claim 1, wherein the swinging unit is comprised of:
a guide body including a main guide member having the first curvature for defining the main track and at least one second guide having the second curvature that is larger than the first curvature for defining the at least one sub-track; and
a belt surrounded on the guide body for holding the transducer unit in order to transmit and receive ultrasounds in an orthogonal direction to each of the main track and the at least one sub-track for swinging the transducer unit along the guide body.

5. The ultrasound probe according to claim 1, wherein the swinging unit includes:
an arm for rotatably hold the transducer unit at one edge of the arm;
a first driving unit connected to the other edge of the arm for swinging the arm within a center angle of an circular arc for defining the main track;
a pulley fixed to the transducer unit;
a belt surrounded on the pulley; and
a second driving unit for swinging the transducer unit so as to perform ultrasound transmissions/receptions in the orthogonal direction to the main track by stopping the belt and for performing ultrasound transmissions/receptions in the orthogonal direction to the sub-track by driving the belt.

6. The ultrasound probe according to claim 1, wherein the transducer unit includes a plurality of transducers arranged in an array so as to perform ultrasound transmissions/receptions in the orthogonal direction to the main track and the at least one sub-track in order to scan ultrasounds in the orthogonal direction to the direction of the to the ultrasound transmissions/receptions.

7. An ultrasound probe comprising:
a transducer unit including a plurality of transducers arranged in an array so as to transmit and receive ultrasounds to and from an object; and
a swinging unit configured to swing the transducer unit in an orthogonal direction to the array of the plurality of transducers for swinging the transducer unit along a straight main track and at least one curved sub-track member having a prescribed curvature, and connected to an edge of the straight main track.

8. An ultrasound diagnosis apparatus comprising:
a transducer unit configured to transmit and receive ultrasounds to and from an object;
an ultrasound probe having a swinging unit configured to swing the transducer unit along a defined main track having a first curvature and a defined at least one sub-track having a second curvature that is larger than the first curvature, the at least one sub-track is connected to the main track;
an image data generating unit configured to generate 3-D image data in a scope of the main track and the at least one sub-track based on the received ultrasound data through the transducer unit; and
a system control unit configured to control whole operations of each of the units in the ultrasound diagnosis apparatus.

9. The ultrasound diagnosis apparatus according to claim 8, wherein the swinging unit includes:
a detection unit configured to detect a swinging angle of the arm rotatably holds the transducers unit;
whereby the system control unit calculates the swinging angle based on the swinging angle data detected by the detection unit and controls the swinging unit based on the calculated swinging angle.

10. An ultrasound diagnosis apparatus comprising:
a transducer unit configured to transmit and receive ultrasounds to and from an object;
an ultrasound probe having a swinging unit configured to swing the transducer unit along a defined straight main track and a defined at least one sub-track having a prescribed curvature and connected to the main track;
a swinging unit configured to swing the transducers unit along the straight main track and the curved sub-track connected to the straight main track;
an image data generating unit configured to generate 3-D image data in a scope of the main track and the curved sub-track based on the received ultrasound data through the transducer unit; and
a system control unit configured to control whole operations of each unit in the ultrasound diagnosis apparatus.
